(19) United States
(12) Patent Application Publication
(10) Pub. No.: US 2011/0288378 A1
(43) Pub. Date: Nov. 24, 2011

(54) Method of Administering A Lifestyle Tracking System

(76) Inventors: Timothy D. Codd, Brighton, MI (US); Sean D. Bumstead, Clarkston, MI (US)

(21) Appl. No.: 13/114,570

(22) Filed: May 24, 2011

Related U.S. Application Data
(60) Provisional application No. 61/347,730, filed on May 24, 2010.

(51) Int. Cl. A61B 5/00

(52) U.S. Cl. 600/300

(57) ABSTRACT

A method of administering a lifestyle tracking system using a computer and a storage medium connected to the computer is provided herein. For the method, a lifestyle variable is inputted into the computer in response to an action by a user of the tracking system. The inputted lifestyle variable is inputted in the storage medium that is connected to the computer. The inputted lifestyle variable is compared to a pre-set benchmark for the lifestyle variable using the computer, and the computer determines whether the pre-set benchmark is met based upon the inputted lifestyle variable. After comparing the inputted lifestyle variable to the pre-set benchmark, feedback is provided to the user of the tracking system using the computer. The feedback includes a reward upon the computer determining that the pre-set benchmark is met based upon the inputted lifestyle variable.

### RELATED APPLICATIONS

This application claims priority to and all the advantages of U.S. Provisional Patent Application No. 61/347,730, filed on May 24, 2010.

### BACKGROUND OF THE INVENTION

1. Field of the Invention

The instant invention generally relates to a method of administering a lifestyle tracking system using a computer and a storage medium connected to the computer. More specifically, the instant invention relates to a method of administering a lifestyle tracking system that compares a lifestyle variable to a pre-set benchmark.

2. Description of the Related Art

Deficiencies in healthy living have become not only a serious threat to human quality of life, but have wide-ranging effects on society in general. Indeed, the effects of unhealthy lifestyles impact schools, the medical field, law enforcement, municipalities, labor organizations, and the insurance industry. While the incidence of childhood and adult obesity has grown to epidemic levels, a great societal need and desire has arisen to counteract such trends. Many parents are aware of the trends in childhood obesity and have sought ways to prevent their children from developing poor health habits. However, with the fast-paced modern lifestyle, it is often difficult to stay the course and ensure that healthy lifestyle habits are consistently and faithfully followed.

Many efforts have been undertaken to confront the trend toward unhealthy lifestyles, from weight management programs to educational programs that teach healthy lifestyle habits to increased availability of health-conscious children's programming. However, merely providing health-conscious dietary or activity options is insufficient to impress upon individuals the greater long-term benefits associated with engaging in the health-conscious options over less healthy options, which often provide immediate gratification.

With the advent of computer technology and the growth in technological awareness, especially among children, technological approaches have also been developed to assist children and adults with maintaining healthy lifestyle habits. In particular, computer programs have been developed that track diet and/or body metrics over time. However, once the novelty wears off, adherence to usage of such programs generally wanes due to the time commitment and diligence that is necessary to properly update the program. With the benefits to adhering to such programs realized only after an extended period of time (or, in some cases, never realized), such programs can become just another unused icon on a personal computer. Furthermore, existing programs are unlikely to capture sufficient information regarding lifestyle habits to effectively assess the general health of the user of the program. For example, if a program tracks eating habits, even if healthy eating habits are followed, other lifestyle choices made by the user (such as lack of activity, lack of sleep, lack of diligence with regard to medical issues, etc.) could detract from any positive results that could be realized from the healthy eating habits.

In view of the magnitude of the societal deficiencies in healthy living and the ill-effects that are felt from such deficiencies, and in view of the ineffectiveness of current efforts to shift the trends in the incidence of unhealthy living, there remains an opportunity to develop methodologies that promote healthy living while fostering consistent and faithful adherence over extended periods of time.

### SUMMARY OF THE INVENTION AND ADVANTAGES

A method of administering a lifestyle tracking system using a computer and a storage medium connected to the computer is provided herein. For the method, a lifestyle variable is inputted into the computer in response to an action by a user of the tracking system. The lifestyle variable is inputted in the storage medium that is connected to the computer. The inputted lifestyle variable is compared to a pre-set benchmark for the lifestyle variable using the computer, and the computer determines whether the pre-set benchmark is met based upon the inputted lifestyle variable. After comparing the inputted lifestyle variable to the pre-set benchmark, feedback is provided to the user of the tracking system using the computer. The feedback includes a reward upon the computer determining that the pre-set benchmark is met based upon the inputted lifestyle variable.

Multiple benefits are achieved by providing a reward to users of the program based upon achieving the pre-set benchmark. In particular, users of the tracking system are incentivized to not only make healthy lifestyle choices, but the potential to earn rewards helps to ensure that the tracking system is continually used for an extended period of time, even after the novelty of the system to the user abates. The potential for rewards also helps to ensure that healthy lifestyle habits are consistently and faithfully followed over time, therefore increasing the chances for positive individual results and positive societal impact on a large scale. Furthermore, the method of the instant invention can serve as a basis for revenue generation to organizations, such as schools and non-profit organizations, through external sponsorship of the rewards program by goods and service providers who are seeking to market to the users of the tracking system. Another benefit to the method of the instant invention is the ability to harvest vast quantities of information regarding lifestyle variables chosen by users of the tracking system, which can have value from a marketing or research perspective. Another benefit to the method of the instant invention is that the tracking system can incorporate social networks and connect multiple individuals and entities, such as schools, insurers, physicians, organizations that coordinate activities (e.g., athletic organizations), supermarkets, and restaurants to effectively track multiple lifestyle variables that, together, complete the picture on healthfulness of a user's lifestyle. Another benefit to the method of the instant invention is that the tracking system can actually be used as curriculum in schools, providing for the possibility of converting at least a portion of lunch time to class time through use of the tracking system to study healthy lifestyle habits and the practical effect on health of making various lifestyle choices.

### DETAILED DESCRIPTION OF THE INVENTION

A method of administering a lifestyle tracking system, in accordance with the instant invention, is generally intended for any individual regardless of their current state of health or physical fitness, lifestyle, age, or gender and may be utilized to assist with organizing, tracking, and gauging lifestyle choices that contribute to general health, with a particular focus upon lifestyle choices that contribute to obesity and physical fitness. In one embodiment, use of the tracking system may be required to receive a benefit that is independent from the benefits of use of the system, such as to receive credit for an educational class, discounts on health insurance, coupons, certificates to receive a tangible good, etc. Alternatively, use of the tracking system may be completely voluntary and not connected to any independent benefit outside of the benefits provided by use of the tracking system itself. For example, individuals may choose to use the tracking system for their own personal wellness, or may even choose to use the tracking system to reap rewards such as points, coupons, or certificates for tangible goods, as described in further detail below, which rewards are provided for engaging in healthy lifestyle decisions and attaining pre-set benchmarks. In effect, individuals may use the tracking system for a variety of benefits, not the least of which is to monitor and track lifestyle choices to ensure that a healthy lifestyle is being pursued.

The method of administering the lifestyle tracking system of the instant invention uses a computer and a storage medium that is connected to the computer or network of computers. By “connected”, it is meant that an electronic connection exists between the computer and the storage medium (and other devices that are “connected” to the computer as described in further detail below) such that information can be passed therebetween. To these ends, the “connection” need not necessarily be a physical connection and the connected devices need not be disposed in the same geographic location. The computer generally refers to a processor and the computer with the storage medium connected thereto can be, for example, a personal computer, a laptop computer, a handheld computer, or a tablet computer. Alternatively, the computer that is used in accordance with the method of the instant invention can be located offsite from a user of the tracking system and can be connected to a separate computer being used by the user through various communication channels such as, but not limited to, various internet connections including a wireless connection, cable connection, or telephone line connection. Additionally, it is to be appreciated that multiple computers may be employed and the instant invention is not limited to use of a single computer to perform every step in the method of the instant invention. The computer is typically programmed to perform various steps in the method as described in further detail below.

The storage medium can be any storage medium that is accessible to the computer and may be packaged along with the computer (such as in a laptop, handheld computer, or tablet computer), or may be located separately from the computer. For example, the storage medium can be a centralized record-storage medium utilized by an administrator of the tracking program and accessible to the computer through various communication channels such as, but not limited to, LAN connection, Internet connection, wireless connection, cable connection, and telephone line connection. An electronic visual display is typically connected to the computer and can be, but is not limited to, a computer monitor or a television in combination with a keyboard; a touch screen interface; and combinations thereof.

The method includes the step of inputting a lifestyle variable into the computer in response to an action by a user of the tracking system. The lifestyle variable is further defined as any variable that could have a connection to health or wellness of an individual. In one embodiment, the lifestyle variable is further defined as at least one of a dietary record, an activity record, or a sleep record. For example, the lifestyle variable could represent a food choice or any metric connected therewith, such as type, amount, brand of food, etc. As described in further detail below, the dietary record can even be a record of a food purchase at a grocery store, with the record obtained from a bar code associated with the food purchase. Examples of activity records include exercise activities such as running, biking, swimming, etc. or any metric connected therewith such as distance, time, etc. Other activity records could include more general categories of activities such as participation in an athletic competition, e.g., a soccer match, hockey game, etc. Examples of sleep records are bed times and rise times, total length of nightly sleep, nap times, etc. In another embodiment, the lifestyle variable is further defined as body mass index (BMI) or metrics that are used by the computer to calculate BMI. For example, the metrics associate with BMI may include height, weight, and age. In yet another embodiment, the lifestyle variable is further defined as a variable associated with a medical condition such as, e.g., sleep deprivation, blood pressure, eyesight, hearing, stress test results, etc. It is to be appreciated that the above examples of lifestyle variables are not exhaustive and that numerous other lifestyle variables that would be useful to monitor can readily be identified by those of skill in the art. In one specific embodiment, a combination of lifestyle variables are inputted into the computer including dietary records, activity records, and sleep records, which combination of lifestyle variables essentially touches upon the major variables that affect overall individual health.

As alluded to above, the lifestyle variable is inputted into the computer in response to an action by a user of the tracking system. In one embodiment, an identifier, such as an identification card, a password, a biometric identifier, or key, is associated with the user of the tracking system and the lifestyle variable is inputted into the computer by a goods or services provider, such as a school lunch room, participating restaurant, grocery store, health club, fitness center, or other pre-approved retailer, using information from the identifier. The identifier can be a dedicated card, code, key, etc. that is only intended for use with the tracking system. Alternatively, the identifier can be a multi-purpose card, code, key, etc. that functions within a grocery store rewards program as well as an identifier for the tracking system, or can be a credit card that is enrolled in the tracking system. In one example, also alluded to above, when the lifestyle variable is the dietary record, the dietary record can be a record of a food purchase at a grocery store, restaurant, or school lunch room. In this embodiment, the action taken by the user may be further defined as ordering or presenting a food item to be purchased to a goods or services provider, whereby the goods or services provider enters information regarding the food item into an electronic cash register such as through scanning a bar code. Before or after the purchase, the user of the tracking system may present the identifier to the goods or services provider, with the information regarding the food item purchased being routed to the tracking system and inputted into the computer automatically.

Alternatively, the action taken by the user of the tracking system may be an affirmative act taken to input the lifestyle variable directly into the computer. In this regard, it is to be appreciated that the affirmative act taken by the user to input the lifestyle variable may vary depending upon various factors including the type of lifestyle variable that is inputted into the computer and the particular users for which the tracking system is designed.

Typically, an electronic visual display is connected to the computer, in which case the method includes communicating an input prompt on the electronic visual display to the user of the tracking system. In one embodiment, the input prompt is further defined as a graphical representation of the lifestyle variable. In this embodiment, the graphical representation of the lifestyle variable is not limited to any particular variable, but is preferably a variable that is standardized and that does not require entry of significant amounts of data to by the user. For example, the graphical representation of the lifestyle variable may be a food item such as a lunch option that is presented in a school cafeteria or an activity record such as “running 1 mile”. For simplicity and to appeal to the senses of the user, when the graphical representation of the lifestyle variable is used, the step of inputting the lifestyle variable into the computer may be further defined as moving the graphical representation of the lifestyle variable from one location on the electronic visual display to another location. For example, a screen layout may be configured on the electronic visual display to appear like a cafeteria lunch line, with the graphical representations of available food items for a particular day being presented. The user may click and drag the food item onto their tray to effectively input the food record into their profile. Although not limited to the embodiment immediately described above, the input prompt may comprise a plurality of pre-set inputs and an administrator of the tracking system may select the pre-set inputs to be presented, which is particularly applicable to the scenario in which graphical representations of the lifestyle variable are presented. To explain, administration of the tracking system may be conveniently performed by a school lunch administrator, with graphical representations of food items for any particular day presented in the tracking system.

Of course, other configurations of input prompts are possible, including conventional configurations such as pull-down lists, fillable fields, etc. However, benefits may be associated with input prompts that are novel and kid-friendly so as to promote continued use of the tracking system.

In one embodiment, multiple users are connected within the tracking system, such as through an online portal. The online community may assist with establishing the tracking system in the youth market. The online portal may connect users in a way that allows a personal and private experience for the user. Settings for privacy levels, necessary support resources, advisory blogs, and even expert chat panels can be made available. The online portal may also allow users to customize email and text reminders in order to stay on a healthy living course. An online calendar, calorie counter, dietary planner, and exercise program may be integrated through the online portal. In one embodiment, the method may further include the step of coordinating a group activity involving the lifestyle variable between multiple users, such as through the online portal. For example, healthy activities such as charity walks, bike trips, and other such activities can be coordinated between multiple users through the online portal, with the option available to associate rewards with participation in the healthy activities and with the option available to coordinate competitions between multiple users.

In accordance with the instant method, the inputted lifestyle variable from the user is stored in the storage medium that is connected to the computer. Typically, the storage medium stores lifestyle variables inputted over a period of time for purposes of tracking the user's lifestyle choices and for purposes of generating reports on the user's lifestyle.

Pre-set benchmarks may be employed within the tracking system for purposes of comparing with the inputted lifestyle variables and provide an excellent tool for assisting the user with working toward a healthy lifestyle. As set forth in further detail below, pre-set benchmarks play an integral role in the reward feature of the instant method, with meeting a pre-set benchmark resulting in presentation of a reward. Pre-set benchmarks can vary greatly within the instant system and can be any standard that, when met, is deemed worthy of receiving any reward. For example, the pre-set benchmark may simply be determination of whether a healthy snack or meal choice was made on a given day. Returning to the example of the school lunch room, the pre-set benchmark may be a determination of whether a healthy lunch selection was made, with an affirmative determination resulting in satisfaction of the pre-set benchmark. Alternatively, the pre-set benchmark may be associated with a long-term goal, such as achieving a particular BMI, weight, fitness level, monthly caloric intake, etc.

In one embodiment, the pre-set benchmarks are pre-established by code within the tracking system program. Alternatively, the pre-set benchmarks may be set by an administrator of the tracking system or by users of the tracking system. For example, when the online portal is used, users can set the pre-set benchmarks. When activities are coordinated in the tracking system through the online portal, participation in the activity may represent the pre-set benchmark. Upon a user inputting participation in the activity or logging performance with regard to the activity, the pre-set benchmark would be satisfied.

As alluded to above, in accordance with the instant method, a determination is made as to whether pre-set benchmarks have been met for the inputted lifestyle variable(s). More specifically, in accordance with the instant method, the computer compares the inputted lifestyle variable to the pre-set benchmark for the lifestyle variable and determines whether the pre-set benchmark is met based upon the inputted lifestyle variable. Given the variety in pre-set benchmarks that are possible, the determination can be made immediately or for a period of time.

Feedback is provided to the user of the tracking system using the computer after comparing the inputted lifestyle variable to the pre-set benchmark. In accordance with the instant method, the feedback includes a reward that is provided to the user upon the computer determining that the pre-set benchmark is met based upon the inputted lifestyle variable. As set forth above, the pre-set benchmark may vary widely and, although the ultimate purpose of the tracking system is to establish healthy lifestyle habits and improve health over time, the provision of a reward need not necessarily be tied to long-term health goals. To maintain user interest, rewards can be tied to even small benchmarks (such as choosing a healthy snack, participating in a healthy activity, or sleeping for a targeted period of time). However, because achievement of significant long-term benchmarks should be rewarded with larger rewards than daily benchmarks, a magnitude of the reward may be correlated to the inputted lifestyle variable so as to establish different rewards depending upon the significance of the benchmark. For example, a healthy snack choice on any given day may garner a small reward, while a series of healthy food choices over a period of time may garner a larger reward. One possible methodology for correlating magnitude of the reward with the inputted lifestyle variable is a scoring system to correlate healthy behavior with reward points. The reward points could be used toward coupons, tangible goods, or experiences. Alternatively, the reward itself can be a coupon, a certificate for a tangible good, or an experience (such as an electronic experience, e.g., a new game, cartoon, etc.; tickets to a sporting event, etc.). The coupons, tangible goods, or experiences present a marking opportunity for vendors of goods and/or services and can even provide a revenue stream for the administrator of the tracking system.

In one embodiment of the instant method, as another form of feedback, the computer generates a report of the lifestyle variables inputted over the period of time, and the report provides an assessment of the inputted lifestyle variables compared to the pre-set benchmark. As an example, the report may be a chart or graph, or may be a visual comparison of current status in relation to the pre-set benchmark. To appeal to the senses of young users, themed reports and profile configurations can be provided. For example, the tracking system may feature a home screen including a school locker that is displayed on the electronic visual display for the particular user, with various graphical tools presented therein including report icons, game icons, and icons for entering lifestyle variables.

In the event that the computer identifies a negative trend compared to the pre-set benchmark, or in the event that rewards are not being earned based upon the lifestyle variables being inputted into the computer, the instant method may include providing feedback including an alert. Associated with the negative alert may be a lesson in how to properly address the lifestyle choice that resulted in the identified negative trend.

In one particular embodiment, the feedback provided in accordance with the instant method includes consistent messaging in the multimedia sensory environment of the target users (e.g., school children) to reinforce healthy behaviors. The users may receive verbal messaging from teachers, administrators, and video blogs, and may receive visual messaging through text, email, and graphic communications.

In one embodiment, the lifestyle variable that is inputted into the computer is communicated to a third party, which third party can utilize the information for various purposes. In one embodiment, lifestyle variables that are inputted from multiple users are compiled, genericized to remove data that would identify particular users, and can be sold to vendors of goods and or services for marketing purposes. For example, in the lifestyle variable may be a dietary record or an activity record and, when the third party is a vendor of goods and/or services, the lifestyle variable may provide insight into purchasing habits or consumer preferences. In another embodiment, the lifestyle variable may be the body mass index or a metrics that are used by the computer to calculate body mass index of the user. In this embodiment, the third party may be an insurance entity. Insurance companies have a vested interest in their client's healthy living habits. If child obesity is decreased, then diabetes and congestive heart failure in adults should also decrease over time. Insurers may be apt to support, endorse, and fund the rewards program and/or to make discounted insurance plans available to users that adhere to the tracking system and exhibit healthy living behavior. Although likely requiring a waiver to grant permission, insurers can possibly review the profiles of users of the tracking system wishing to apply for the discounted plans.

As another function of the system, a cookbook feature can also be included that features healthy recipes. The cookbook feature can be incorporated into the system to track the choices made from the cookbook as lifestyle variables. Additionally, ingredients for menu items in the cookbook can be paired with coupons from vendors of goods and/or services, thereby representing another possible marketing opportunity and revenue stream for the administrator of the tracking system, as well as another opportunity to promote healthy lifestyle habits.

The invention has been described in an illustrative manner, and it is to be understood that the terminology which has been used is intended to be in the nature of words of description rather than of limitation. Obviously, many modifications and variations of the present invention are possible in light of the above teachings, and the invention may be practiced otherwise than as specifically described.

What is claimed is:

1. A method of administering a lifestyle tracking system using a computer and a storage medium connected to the computer, said method comprising the steps of:
inputting a lifestyle variable into the computer in response to an action by a user of the tracking system;
storing the inputted lifestyle variable in the storage medium connected to the computer;
comparing the inputted lifestyle variable to a pre-set benchmark for the lifestyle variable using the computer and determining whether the pre-set benchmark is met based upon the inputted lifestyle variable; and
providing feedback to the user of the tracking system using the computer after comparing the inputted lifestyle variable to the pre-set benchmark;
wherein the feedback includes a reward upon the computer determining that the pre-set benchmark is met based upon the inputted lifestyle variable.

2. A method as set forth in claim 1 wherein the lifestyle variable is further defined as at least one of a dietary record, activity record, or a sleep record.

3. A method as set forth in claim 1 wherein the lifestyle variable is further defined as a body mass index or metrics that are used by the computer to calculate body mass index.

4. A method as set forth in claim 1 wherein the lifestyle variable is communicated to a third party.

5. A method as set forth in claim 4 wherein the lifestyle variable is further defined as a body mass index or a metrics that are used by the computer to calculate body mass index and wherein the third party is an insurance entity.

6. A method as set forth in claim 4 wherein the lifestyle variable is further defined as at least one of a dietary record or an activity record and wherein the third party is a vendor of goods and/or services.

7. A method as set forth in claim 1 wherein the storage medium stores lifestyle variables inputted over a period of time.

8. A method as set forth in claim 7 wherein the computer generates a report of the lifestyle variables inputted over the period of time and wherein the report provides an assessment of the inputted lifestyle variables compared to the pre-set benchmark.

9. A method as set forth in claim 1 wherein the feedback includes an alert upon the computer identifying a negative trend compared to the pre-set benchmark.

10. A method as set forth in claim 1 wherein an electronic visual display is connected to the computer and wherein the method further comprises the step of communicating an input prompt on the electronic visual display to the user of the tracking system.

11. A method as set forth in claim 10 wherein the input prompt is further defined as a graphical representation of the lifestyle variable.

12. A method as set forth in claim 11 wherein the step of inputting the lifestyle variable into the computer is further defined as moving the graphical representation of the lifestyle variable from one location on the electronic visual display to another location.

13. A method as set forth in claim 10 wherein the input prompt comprises a plurality of pre-set inputs and wherein an administrator of the tracking system selects the pre-set inputs to be presented.

14. A method as set forth in claim 1 wherein an identifier is associated with the user of the tracking system and wherein the lifestyle variable is inputted into the computer by a goods or services provider using information from the identifier.

15. A method as set forth in claim 1 wherein a magnitude of the reward is correlated to the inputted lifestyle variable.

16. A method as set forth in claim 1 wherein multiple users are connected within the tracking system and wherein the method further comprises the step of coordinating a group activity involving the lifestyle variable between multiple users.
